(19) United States
(12) Patent Application Publication
(10) Pub. No.: US 2004/0147908 A1
(43) Pub. Date: Jul. 29, 2004

(54) Accessory for over the wire catheter with short wire capability

(76) Inventor: Niall Duffy, Galway (IE)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/350,637

(22) Filed: Jan. 24, 2003

(51) Int. Cl. A61B 17/00

(52) U.S. Cl. 606/1

(57) ABSTRACT

The present invention is an accessory for use with an over the wire catheter with short wire capability having an elongated shaft, a guide wire lumen and a guide member associated with the elongated shaft for providing transverse access to the guide wire lumen through a passageway. The accessory includes a first member for securing the accessory to the guide member and a second member having a passageway that aligns with the guide member passageway. The accessory may be used for front-loading a guide wire through the guide member. Alternatively, the accessory may be used to attach a syringe to assist in flushing the guide wire lumen through the guide member.

### FIELD OF THE INVENTION

The present invention relates to catheters used with guide wires in the cardiovascular system and more particularly to an accessory that assists guide wire loading and flushing of the catheter guide wire lumen.

### BACKGROUND OF THE INVENTION

10021 Catheters are inserted to various locations within a patient for a wide variety of purposes and medical procedures. For example only, one type of catheter is used in percutaneous catheter intervention (PCI) for the treatment of a vascular constriction termed a stenosis. In this instance, the catheter has a distally mounted balloon that can be placed, in a deflated condition, within the stenosis, and then inflated to dilate the narrowed lumen of the blood vessel. Such balloon dilation therapy is generally named percutaneous transluminal angioplasty (PTA). The designation PTCA, for percutaneous transluminal coronary angioplasty, is used when the treatment is more specifically employed in vessels of the heart. PTCA is used to open coronary arteries that have been occluded by a build-up of cholesterol fats or atherosclerotic plaque. The balloon at the distal end of the catheter is inflated, causing the site of the stenosis to widen.

The dilation of the occlusion, however, can form flaps, fissures and dissections, which may result in reclosure of the dilated vessel or even perforations in the vessel wall. Implantation of a stent can provide support for such flaps and dissections and thereby prevent reclosure of the vessel or provide a patch repair for a perforated vessel wall until corrective surgery can be performed. A stent is typically a cylindrically shaped device formed from wire(s) or a metal tube and is intended to act as a permanent prosthesis. A stent is deployed in a body lumen from a radially compressed configuration into a radially expanded configuration that allows it to contact and support a body lumen. A stent can be implanted during an angioplasty procedure by using a balloon catheter bearing a compressed stent that has been loaded onto the balloon. The stent radially expands as the balloon is inflated, forcing the stent into contact with the body lumen, thereby forming a supporting relationship with the lumen walls. Alternatively, self-expanding stents may be deployed with a sheath-based delivery catheter. Deployment is effected after the stent has been introduced percutaneously, transported transluminally and positioned at a desired location by the delivery catheter. In addition to angioplasty and stenting procedures, other therapeutic procedures require use of a delivery catheter, such as drug delivery, filters, occlusion devices, diagnostic devices and radiation treatment.

Typically, the placement of such therapeutic delivery catheters involves the use of a guide wire, which may be inserted into the patient's vasculature through the skin, and advanced to the location of the treatment site. The delivery catheter, which has a lumen adapted to receive the guide wire, then is advanced over the guide wire. Alternatively, the guide wire and the delivery catheter may be advanced together, with the guide wire protruding from the distal end of the delivery catheter. In either case, the guide wire serves to guide the delivery catheter to the location to be treated.

There are four general types of catheters: “over-the-wire” (OTW) catheters, “over-the-wire catheters with short wire capability “(OTW/SW) such as disclosed in U.S. Pat. No. 4,998,356 (Crittenden, et al.) and co-pending application U.S. Ser. No. 10/116,234, “rapid exchange” catheters and “fixed wire” or “a balloon on a wire” catheters. OTW and rapid exchange catheters require use of a guide wire separate from the catheter while a fixed wire or balloon on a wire catheter has an integral guide wire. An OTW catheter comprises a guide wire lumen that extends the entire length of the catheter. The guide wire is disposed entirely within the catheter guide wire lumen except for distal and proximal portions of the guide wire, which extend beyond the distal and proximal ends of the catheter respectively. An OTW/SW catheter has an over-the-wire configuration while the catheter is within the patient's body. Thus, the guide wire is disposed entirely within the catheter guide wire lumen, except for the distal and proximal portion of the guide wire, which extend beyond the distal and proximal ends of the catheter respectively when it is fully inserted into the patient.

OTW and OTW/SW catheters have many advantages traceable to the presence of the full length guide wire lumen, such as good stiffness and pushabilty for readily advancing the catheter through the tortuous vasculature and across tight stenosis. The full-length guide wire lumen permits removing and replacement of a guide wire in an indwelling catheter, as may be required to alter the shape of the guide wire tip. It is also sometimes desirable to exchange one guide wire for another guide wire having a different stiffness. For example, a relatively soft, or flexible guide wire may prove to be suitable for guiding a PTCA catheter through a particular tortuous anatomy, whereas following up with a stent deliver catheter through the same vasculature region may require a guide wire that is relatively stiffer.

Traditional over-the-wire catheters do suffer some shortcomings, however. For example, it often becomes necessary, in the performance of a PCI, to exchange one indwelling catheter for another catheter. In order to maintain a guide wire in position while withdrawing the catheter, the guide wire must be gripped at its proximal end to prevent it from being pulled out of the blood vessel with the catheter. For example, a PTCA catheter, which may typically be on the order of 135 centimeters long, is longer than the proximal portion of the standard guide wire that protrudes out of the patient. Therefore, exchanging an over-the-wire PTCA catheter requires an exchange guide wire of about 300 centimeters long, whereas a standard guide wire is about 165 centimeters long.

In one type of over-the-wire catheter exchange, the standard length guide wire first is removed from the lumen of the indwelling catheter. Then, a longer exchange guide wire is passed through the catheter to replace the original wire. Next, while holding the exchange guide wire by its proximal end to control its position in the patient, the catheter is withdrawn proximally from the blood vessel over the exchange guide wire. After the first catheter has been removed, the next OTW catheter is threaded onto the proximal end of the exchange guide wire and is advanced along the exchange guide wire, through the guiding catheter, and into the patient's blood vessels until the distal end of the catheter is at the desired location. The exchange guide wire may be left in place or it may be exchanged for a shorter, conventional-length guide wire. In an alternative type of catheter exchange procedure, the length of the initial guide wire may be extended by way of a guide wire extension apparatus. Regardless of which exchange process is used, the very long exchange guide wire is awkward to handle, thus requiring at least two operators to perform the procedure.

An OTW catheter designed to eliminate the need for guide wire extensions or exchange wires is disclosed in U.S. Pat. No. 4,988,356 (Crittenden et al.). This OTW/SW catheter includes a catheter shaft having a cut that extends longitudinally between the proximal end and the distal end of the catheter and that extends radially from the catheter shaft outer surface to the guide wire lumen. A guide member slidably coupled to the catheter shaft functions to open the cut such that the guide wire may extend transversely into or out of the cut at any location along its length. By moving the guide member, the effective over-the-wire length of the OTW/SW catheter is adjustable.

When using the OTW/SW catheter, the guide wire is maneuvered through the patient's vascular system such that the distal end of the guide wire is positioned across the treatment site. With the guide member positioned near the distal end of the catheter, the proximal end of the guide wire is threaded into the guide wire lumen opening at the distal end of the catheter and through the guide member such that the proximal end of the guide wire protrudes out the proximal end of the guide member. By securing the guide member and the proximal end of the guide wire in a fixed position, the catheter may then be transported over the guide wire by advancing the catheter toward the guide member. In doing so, the catheter advances through the guide member such that the guide wire lumen envelops the guide wire as the catheter is advanced into the patient's vasculature. In a PTCA embodiment, the OTW/SW catheter may be advanced over the guide wire in this manner until the distal end of the catheter having the dilatation balloon is positioned within the stenosis and essentially the entire length of the guide wire is encompassed within the guide wire lumen.

Furthermore, the indwelling OTW/SW catheter may be exchanged with another catheter by reversing the operation described above. To this end, the indwelling catheter may be removed by withdrawing the proximal end of the catheter from the patient while holding the proximal end of the guide wire and the guide member in a fixed position. When the catheter has been withdrawn to the point where the distal end of the cut has reached the guide member, the distal portion of the catheter over the guide wire is of a sufficiently short length that the catheter may be drawn over the proximal end of the guide wire without releasing control of the guide wire or disturbing its position within the patient. After the catheter has been removed, another OTW/SW catheter may be threaded onto the guide wire and advanced over the guide wire in the same manner described above with regard to the OTW/SW catheter. The OTW/SW catheter permits catheter exchange without the use of the very long exchange guide wire.

Guide wires are commonly back loaded into the delivery catheter. In this operation, the guide wire proximal end is inserted into the distal tip of the catheter. It is pushed through the catheter until it extends out of the proximal guide wire exit. In a traditional over-the-wire catheter the proximal guide wire exit is the proximal end of the catheter through its inflation luer. The rapid exchange proximal guide wire exit is the termination of the short guide wire tube a few centimeters or typically 25 centimeters beyond the distal tip of the catheter. In the OTW/SW catheter, the proximal guide wire exit is through the guide member positioned on the proximal shaft of the catheter. As an alternative to back loading a guide wire into the delivery system, a guide wire may also be front-loaded. In a front-loading operation, the distal tip of the guide wire is inserted into the guide wire lumen on the proximal shaft and pushed through until it exits the distal tip of the delivery catheter. A front-loading operation is possible with OTW and OTW/SW catheters if the guide wire will be exchanged during procedures. A front loading operation is not used with a rapid exchange catheter since the guide wire cannot be exchanged while the catheter is inserted into the patient. With a rapid exchange catheter, the insertion of the distal tip into the proximal end of the guide wire lumen is pure chance due to the fact that the proximal end is typically 125 centimeters from the exit location of the catheter from the patient at the femoral artery in the groin.

In an over-the-wire catheter with short wire capability the front-loading procedure will occur through the guide member and become only associated with the exchanging of the guide wire during a procedure. The distal tip of a guide wire is extremely flexible and small and thus may be difficult to thread into the guide member of the over-the-wire with short guide wire capability catheter. In order to improve procedural times, it is preferable to have an easy way to insert the guide wire through a front-loading procedure. Accordingly, the present invention addresses the loading of a guide wire through the guide member of the over-the-wire catheter with short guide wire capability.

Guide wire lumens are often flushed prior to procedure. Additionally, there may be a situation where the delivery catheter is removed, but will then be reinserted into the patient for a follow-up inflation of the balloon at the treatment site. Accordingly, the practitioner will also desire to flush the guide wire lumen prior to the reinsertion. One common practice is to insert a syringe or flushing cannula at the distal end of the catheter and thus flush the guide wire lumen from the distal end and remove any blood to prevent coagulation or particulate upon reinsertion of the catheter into the patient. Accordingly, the present invention also assists the practitioner in flushing the proximal guide wire lumen of an OTW/SW catheter.

### SUMMARY OF THE INVENTION

The present invention is a wire loading and guide wire lumen flushing accessory for use with an OTW/SW catheter. The OTW/SW catheter comprises an elongate flexible catheter having proximal and distal ends and first and second lumens extending there through. The first lumen is sized and shaped to receive a guide wire. The second lumen is an inflation lumen. A guide member is mounted on the catheter proximal shaft and enables transverse access to the first lumen through the elongate flexible catheter. A balloon is mounted about catheter distal segment, with the balloon being in fluid communication with the second lumen. The wire loading tool and guide wire lumen flushing accessory has an arcuate section configured to surround the exterior of the guide member. A conical member extends from the arcuate section forming a passageway there through. The passageway tapers from the proximal end to the distal end. When the tool is coupled to the guide member, the passageway is aligned with the guide wire lumen opening in the guide member and thus enables transverse access to the first lumen. The conical member is threaded at its proximal portion to mate with a conventional syringe to enable the practitioner to flush the first lumen through the transverse access provided by the guide member.

### BRIEF DESCRIPTION OF THE DRAWINGS

These and other features, aspects and advantages of the present invention will become better understood with reference to the following description, appended claims, and accompanying drawings where:

FIG. 1 is an illustration of a OTW/SW catheter and guide wire in an assembled configuration;

FIG. 2 is a perspective view of the accessory of the present invention;

FIG. 3 is a plan view of the accessory of the present invention;

FIG. 4 is a side view of the accessory of the present invention;

FIG. 5 is a cross section view of the accessory of the present invention;

FIG. 6 is an illustration of the use of the accessory for guide wire loading prior to securing the accessory to the OTW/SW catheter guide member;

FIG. 7 is an illustration of the use of the accessory for guide wire loading secured to the OTW/SW catheter guide member

FIG. 8 is an illustration of the use of the accessory for flushing the guide wire lumen prior to securing the accessory to the OTW/SW catheter guide member; and

FIG. 9 is an illustration of the accessory and a syringe secured to the guide member in order to flush the guide wire lumen.

### DETAILED DESCRIPTION OF THE INVENTION

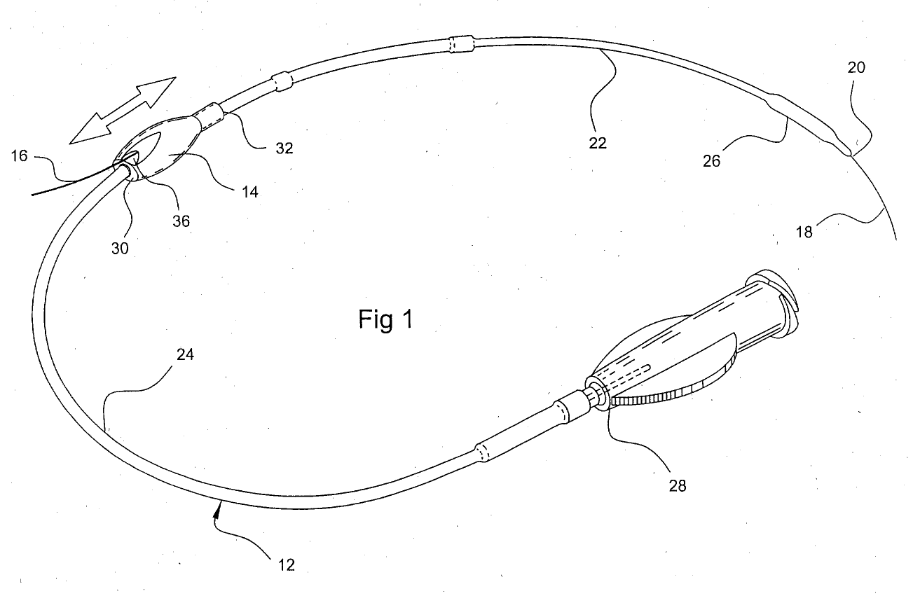

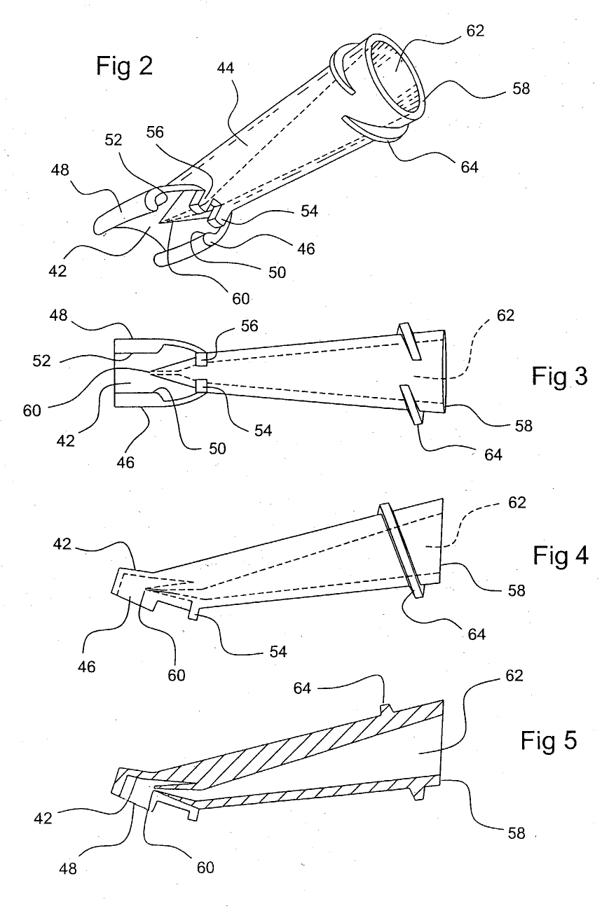

The present invention is a guide wire loading and guide wire lumen flushing accessory 10 shown in FIGS. 2-9 for use with an over the wire catheter having short wire capability (OTW/SW catheter). Turning now to FIG. 1, OTW/SW catheter 12 will first be described. OTW/SW catheter 12 includes guide member 14 with guide wire 16 illustrated as extending through the guide member 14. Guide member 14 serves as a juncture in which the catheter 12 and guide wire 16 may be merged or separated so that the portion of guide wire 16 which extends proximally of guide member 14 (to the left as seen in FIG. 1) is separated from catheter 12 and the portion of guide wire 16 which is located distally of guide member 14 (to the right as seen in FIG. 1) is contained and housed within catheter 12 except for distal end 18 of guide wire 16 which may protrude distally out of distal end 20 of catheter 12.

Catheter 12 includes an elongate, flexible, cylindrical main body, which may be formed from an extruded plastic material such as, for example, polyethylene or polyethylene block amide (PEBA) copolymer. Catheter 12 has a distal shaft 22 and a proximal shaft 24. In the embodiment shown in FIG. 1, catheter 12 is a delivery catheter, such as for PTCA or stent delivery, having balloon 26 mounted around the catheter body near catheter distal end 20. Balloon 26 may be inflated and deflated through an inflation lumen formed through the body of the catheter 12. The inflation lumen extends from the proximal end of catheter 12, where it communicates with fitting 28 and extends the length of catheter 12, terminating in communication with the interior of balloon 26. Fitting 28 may be connected to a suitable source of pressurized fluid or a partial vacuum to inflate or deflate balloon 26. Catheter 12 includes another lumen, which is intended to receive guide wire 16. The guide wire lumen extends the full length of catheter 12, terminating at distal end 20 and proximal fitting 28.

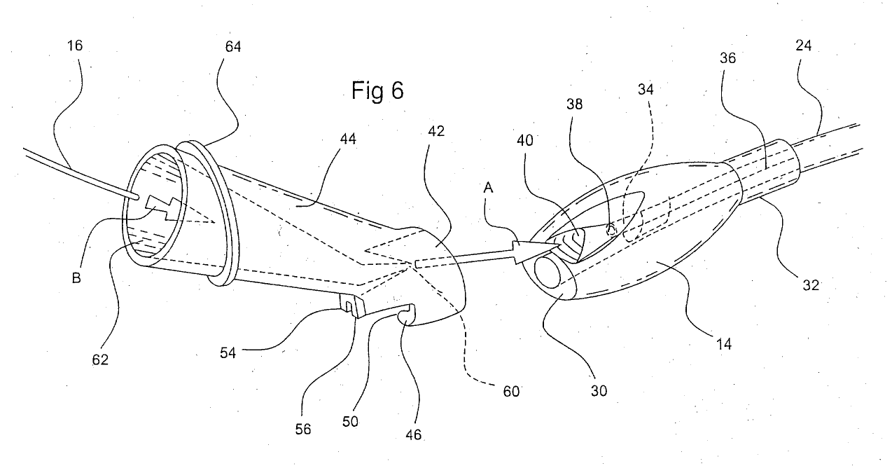

Guide member 14 has proximal and distal ends 30 and 32, respectively, and surrounds proximal shaft 24 as shown in FIGS. 2-9. A guide wire passageway 34 extends distally through the guide member 14 and into guide wire lumen 36 from its proximal end 38 located on guide member 14 (FIGS. 6 and 9). Passageway proximal end 38 is positioned in recess 40 of guide member 14.

Turning now to FIGS. 2-9, the present invention, accessory 10, will be described in detail. Accessory 10 includes a first member, an arcuate plate 42, and a second member, conical member 44. Two arcuate arms 46 and 48 extend from plate 42 at the distal end of plate 42. Arms 46 and 48 contain rimmed edges 50 and 52. Two tabs, 54 and 56, extend from the proximal section of plate 42. Arcuate plate 42, arms 46 and 48 and tabs 54 and 56 are configured to mate with the outer surface of guide member 14 for a snap fit as will be described further below. Conical member 44 tapers from its proximal end 58 to its distal end 60 positioned within the cavity formed by arcuate plate 42. Passageway 62, extending through conical member 44, also tapers from proximal end 58 to distal end 60. Conical member 44 is secured to distal plate 42 just proximal of its distal end 60 as shown. Conical member 44 preferably contains threads 64 that will mate with a conventional syringe as will be explained when the use of accessory 10 is described.

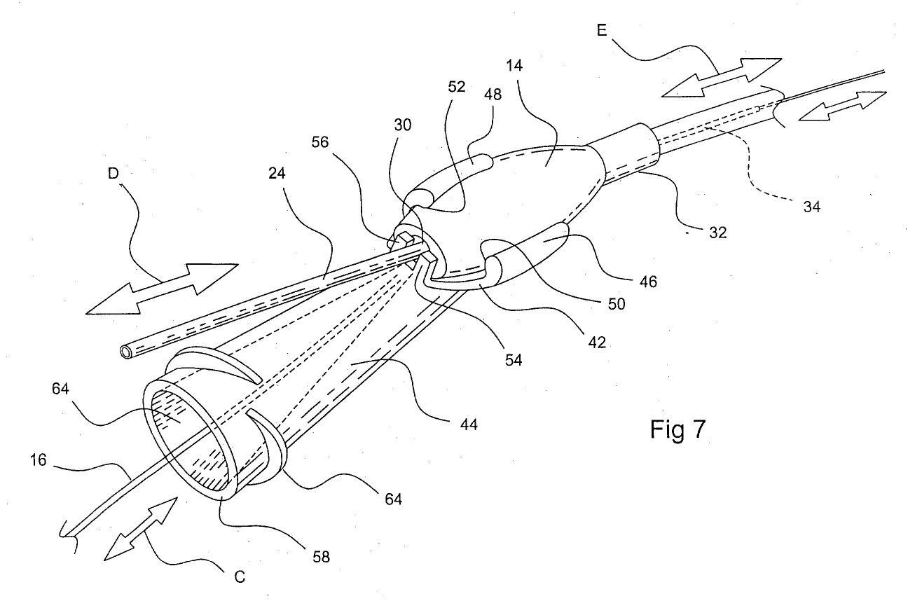

Accessory 10 is used for either loading a guide wire into OTW/SW catheter 12 through the guide member 14 or for attaching a syringe to guide member 14 to assist in flushing the catheter guide wire lumen. Loading a guide wire with accessory 10 will first be described. As shown in FIG. 6, accessory 10 is placed onto guide member 14 such that distal end 60 of conical member 44 is inserted into recess 40 as illustrated by arrow A. Plate 42 will extend over the exterior surface of guide member 14 with arms 46 and 48 wrapping around the sides of guide member 14. Tabs 54 and 56 are positioned against proximal end 30 of guide member 14 and the spacing between tabs 54 and 56 permit proximal shaft 24 of OTW/SW catheter 12 to pass there through as seen in FIG. 7. Accessory 10 preferably couples to guide member 14 with a snap fit that is secure while attached, but is easily removable by the user. Once in position on guide member 14, distal tip 60 is positioned aligned and within guide wire passageway 34 and accessory 10 is ready to use. The slope of passageway 62 is preferably selected to match the slope of guide member passageway 34 such that a smooth transition results. The guide wire tip will enter catheter 12 through a smooth passageway and will not be damaged as it is introduced through accessory 10 and into catheter 12.

Guide wire 16 is directed into passageway 62 as illustrated by arrow B in FIG. 6. Tapered passageway 62 directs the distal tip of guide wire 16 into guide wire passageway 34 and into guide wire lumen 36 of OTW/SW catheter 12. As shown in FIG. 7, guide wire 16 is movable distally and proximally when accessory 10 is in position on guide member 14 as illustrated by arrow C in FIG. 7. Additionally, accessory 10 does not hinder movement of catheter proximal shaft 24 through guide member 14 as depicted by arrows D and E in FIG. 7. Once guide wire 16 has been loaded, accessory 10 may be uncoupled from guide member 14 and pulled proximally over the proximal end of guide wire 16 to remove it from the OTW/SW catheter 12.

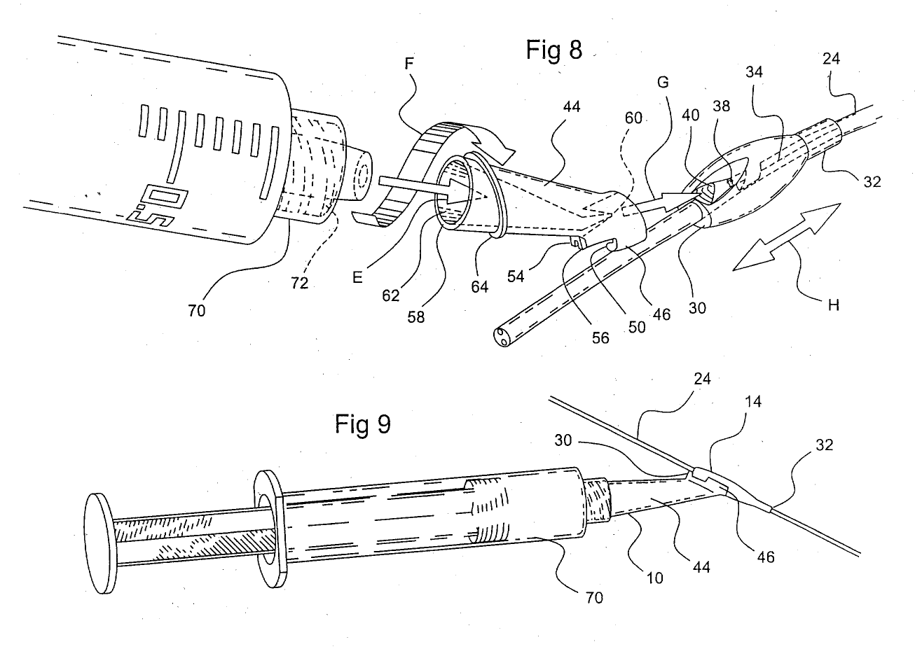

Accessory 10 may also be used to assist the practitioner in flushing the guide wire lumen. Conventional syringe 70 is attached to accessory 10 as illustrated by arrows E and F in FIG. 8. Threads 64 mate with threads 72 on syringe 70 to fully secure syringe 70 and accessory 10. Accessory 10 is secured to guide member 14 as illustrated by arrow G in FIG. 8. Once syringe 70 is attached to accessory 10 and accessory 10 is attached to guide member 14 as shown in FIG. 9, guide wire lumen 36 is ready to be flushed. Guide member 34, moveable with respect to proximal shaft 24 as illustrated by arrow H, is preferably positioned at its most proximal position on proximal shaft 24 of OTW/SW catheter 12. While holding catheter 12, guide member 14 is advanced to its most extended position on the proximal shaft of the catheter while depressing syringe to deploy flushing fluid from syringe 70 into passageway 62, guide wire passageway 34 and into guide wire lumen 36. Once guide member 14 has reached its distal position on the proximal shaft and guide wire lumen 36 is sufficiently flushed, syringe 70 and accessory 10 may be removed from guide member 14.

While the invention has been particularly shown and described with reference to the preferred embodiments thereof, it will be understood by those skilled in the art that various changes in form and detail may be made there in without departing from the spirit and scope of the invention.

What is claimed is:

1. An accessory for use with an over the wire catheter with short wire capability having an elongated shaft, a guide wire lumen and a guide member associated with the elongated shaft for providing transverse access to the guide wire lumen through a passageway; the accessory comprising:
a. a first member for securing the accessory to the guide member;
b. a second member extending from the first member and containing a passageway that aligns with the guide member passageway.

2. An accessory of claim 1 wherein the second member has a proximal end and a distal end and the second member passageway tapers from the proximal end to the distal end.

3. An accessory of claim 2 wherein the second member distal end is received within the guide member passageway.

4. An accessory of claim 1 wherein the second member contains means for coupling the accessory to a syringe.

5. An accessory of claim 4 wherein the coupling means is threads.

6. An accessory of claim 2 wherein the second member passageway is in fluid communication with the guide member passageway.

7. An accessory of claim 1 and further comprising the guide member having an arcuate exterior surface wherein the first member is an arcuate plate having arcuate arms extending there from that correspond to the arcuate exterior of the guide member.
